(19) United States
(12) Patent Application Publication
(10) Pub. No.: US 2024/0208944 A1
(43) Pub. Date: Jun. 27, 2024

(54) Quinazoline Derivatives, Pharmaceutical Compositions, and Therapeutic Uses Related to Nox Inhibition

(71) Applicants: Emory University, Atlanta, GA (US); The Medical College of Wisconsin, Inc., Milwaukee, WI (US)

(72) Inventors: Thota Ganesh, Cumming, GA (US); Radhika Amaradhi, Decatur, GA (US); Jacek Zielonka, Wauwatosa, WI (US)

(21) Appl. No.: 18/287,632

(22) PCT Filed: Apr. 19, 2022
(86) PCT No.: PCT/US2022/025261

§ 371 Date: Oct. 19, 2023

Related U.S. Application Data
(60) Provisional application No. 63/176,699, filed on Apr. 19, 2021.

(51) Int. Cl. C07D 403/12; A61K 31/517; C07D 235/28; C07D 401/12

(52) U.S. Cl. CPC C07D 403/12; A61K 31/517; C07D 235/28; C07D 401/12

(57) ABSTRACT

The disclosure relates to quinazoline derivatives, pharmaceutical compositions, and therapeutic methods related thereto. In certain embodiments, this disclosure relates to compounds and methods of treating or preventing a Nox-related disease comprising administering to a subject a pharmaceutical composition comprising a Nox inhibitor or derivative reported herein.

### CROSS-REFERENCE TO RELATED APPLICATIONS

This application claims the benefit of U.S. Provisional Application No. 63/176,699 filed Apr. 19, 2021. The entirety of this application is hereby incorporated by reference for all purposes.

### STATEMENT REGARDING FEDERALLY SPONSORED RESEARCH OR DEVELOPMENT

This invention was made with government support under AI110680 awarded by the National Institues of Health. The government has certain rights in the invention.

### BACKGROUND

The NADPH oxidases (Nox) are a family of enzymes expressed in various cell types and generate superoxide which secondarily results in other reactive oxygen species, including hydrogen peroxide. These reactive oxygen species (ROS) cause cell damage and inflammation. Nox enzymes are implicated in a wide range of diseases. Ma et al. report NADPH oxidase has implications in brain injury and neurodegenerative disorders such as stroke, traumatic brain injury (TBI), Alzheimer's disease (AD), Parkinson's disease (PD), Huntington's disease (HD), amyotrophic lateral sclerosis (ALS), and multiple sclerosis (MS). Mol Neurodegener, 2017, 12, 7. See also McBean et al., Br J Pharmacol, 2017, 174(12): 1750-1770. Nox enzymes inhibitors have been reported; however, there exists a need to identify improvements.

Zielonka et al. report high-throughput assays for superoxide and hydrogen peroxide: design of a screening workflow to identify inhibitors of NADPH oxidases. J Biol Chem, 2014, 289(23): 16176-89.

Li et al. report thioxo-dihydroquinazolinone compounds as inhibitors of myeloperoxidase. ACS Med Chem Lett, 2015, 6(10): 1047-52.

References cited herein are not an admission of prior art.

### SUMMARY

It has been discovered that certain compounds inhibit Nox enzymes. In some embodiments, this disclosure relates to compounds and methods of treating or preventing a Nox-related disease comprising administering to a subject a pharmaceutical composition comprising a Nox inhibitor or derivative such as a compound of formula I,

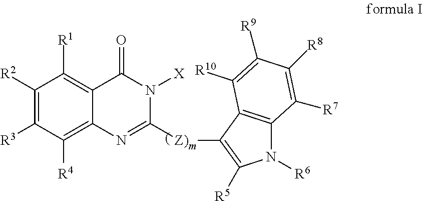

or pharmaceutically acceptable salt, prodrug or derivative thereof, wherein substituents are reported herein. In certain embodiments, the derivatives may be any compound disclosed herein optionally substituted with one or more, the same or different, substituents or salts thereof.

In certain embodiments, the disclosure relates to the use of a compound as described herein in the production of a medicament for the treatment of a Nox related disease. Compounds disclosed here can be contained in pharmaceutical compositions and administered alone or in combination with one or more additional active agents. The active agents can be administered simultaneously in the same dosage form or in separate dosage forms. Alternatively, the active agents can be administered sequentially in different dosage forms.

The compound can be combined with one or more pharmaceutically acceptable excipients to form a pharmaceutical composition. The compositions can be formulated for enteral, parenteral, topical, transdermal, or pulmonary administration.

The compounds described herein can be used to treat a variety of Nox-related diseases including, but not limited to, neurodegenerative disorders, stroke, traumatic brain injury (TBI), Alzheimer's disease (AD), Parkinson's disease (PD), Huntington's disease (HD), amyotrophic lateral sclerosis (ALS), and multiple sclerosis (MS), central nervous system disorders, hypertension, chronic obstructive pulmonary disease (COPD), acute respiratory distress syndrome (ARDS), acute lung injury (ALI), atherosclerosis, aging-related deafness, inflammatory diseases, such as arthritis; various cancers such as colon cancer, prostate cancer, fibrotic diseases, such as liver fibrosis, pulmonary fibrosis, idiopathic pulmonary fibrosis, cirrhosis, endomyocardial fibrosis, mediastinal fibrosis, myelofibrosis, retroperitoneal fibrosis, nephrogenic systemic fibrosis, Crohn's disease, and scleroderma/systemic reperfusion injury-related disorders, such as myocardial infarction; ischemic stroke, preservation of organs during transplantation, ischemia/reperfusion injury (including stroke, myocardial infarction), diabetes, acute lung inflammation, cardiac hypertrophy, diabetic nephropathy, scar formation, skin aging and damage, and psoriasis.

In some embodiments, it is contemplated that compositions disclosed herein can be administered to subject before, during or after certain medical procedures, such as, organ transplants (heart, kidneys, liver, lungs, pancreas, intestine, and thymus) or other surgeries that reduce blood flow (cardiovascular surgery).

In some embodiments, it is contemplated that composition disclosed herein can be used in biological (organ, tissue, or cell) storage mediums, typically aqueous solutions maintained at or below room temperatures, which may contain other ingredients such as, but not limited to, salts (sodium chloride, sodium lactate, calcium chloride, potassium chloride), amino acids, saccharides, polysaccharides (dextran, chondroitin, hydroxyethyl starch), vitamins (thiamine, ascorbic acid, calciferol, riboflavin, pyridoxine, tocopherol, cobalamins, phylloquinone, pantothenic acid, biotin, niacin, folic acid) and/or adenosine triphosphate or precursors (adenosine, inosine, and adenine).

In certain embodiments, the disclosure relates to method of making compounds disclosed herein by mixing starting materials and reagents disclosed herein under conditions such that the compounds are formed.

### BRIEF DESCRIPTION OF THE DRAWING(S)

FIG. 1A illustrates the structures of certain compounds of interest.

FIG. 1B shows data of pharmaceutical candidates identified from Nox assays.

FIG. 2A shows concentration of TG15-132S over time after IP injection of 20 mg/kg in male SD rats. Compound displays half-life of 3.7 h in plasma and 5.6 h in the brain.

FIG. 2B shows in vivo rat pharmacokinetics of TG15-132S. The compound has a brain-to-plasma ratio >10.

FIG. 3 illustrates contemplated chemical formulas III, IV, and V wherein HX is any inorganic or organic salt, including HCI, HBr, HSO₄, oxalate, malate, etc; n is 0, 1, 2, or 3; X, Y are each individually and independently hydrogen, halogen, nitro, amino, aminoalkyl, dialkylamino, aliphatic, aromatic and heteroaromatic rings optionally substituted; Ar refers to any heterocyclic ring, optionally substituted aromatic and heteroaromatic, or aliphatic cyclic ring structure; Z is S, NH, O, NR, where R═H, alkyl and aryl groups optionally substituted.

FIG. 4 illustrates additional embodiments of this disclosure.

FIG. 5 illustrates additional embodiments of this disclosure. Each R may be independently and individually selected from hydrogen, alkyl, halogen, hydroxy, carbocyclyl, aryl, or heterocyclyl each of which may be optionally substituted with a substituent.

FIG. 6 illustrates additional embodiments of this disclosure.

FIG. 7 shows data where THP1 moocytic cells were treated with PMA 100 ng/ml to induced mRNA levels of NOX2 (gp91phox) and its catalytic subunits (p47phox, p67phox) and inflammatory markers IL-1beta and TNF. These markers are suppressed by the treatment of NOX2 inhibitor TG15-132.HCI.

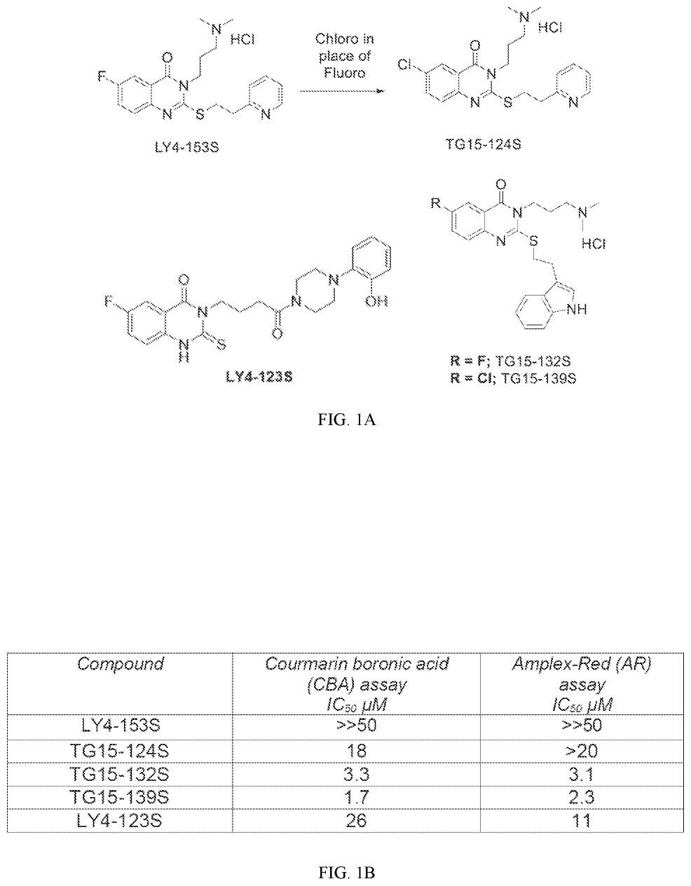

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to an assertion that such a disclosure was a disclosure of the inventors, derived from the inventors, or antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise.

As used herein, “alkyl” means a noncyclic straight chain or branched, unsaturated or saturated hydrocarbon such as those containing from 1 to 10 carbon atoms, typically 1 to 6 carbon atoms. Within any embodiments, herein alkyl may refer to an alkyl with 1 to 6 carbons (C₁₋₆alkl). Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“Aryl” means an aromatic carbocyclic monocyclic or polycyclic ring such as phenyl or naphthyl. Polycyclic ring systems may, but are not required to, contain one or more non-aromatic rings, as long as one of the rings is aromatic.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

“Heterocarbocycles” or heterocarbocyclyl” groups are carbocycles which contain from 1 to 4 heteroatoms independently selected from nitrogen, oxygen and sulfur which may be saturated or unsaturated (but not aromatic), monocyclic or polycyclic, and wherein the nitrogen and sulfur heteroatoms may be optionally oxidized, and the nitrogen heteroatom may be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

As used herein, “heteroaryl” refers an aromatic heterocarbocycle having 1 to 4 heteroatoms selected from nitrogen, oxygen and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent. “Alkylthio” refers to an alkyl group as defined above with the indicated number of carbon atoms attached through a sulfur bridge. An example of an alkylthio is methylthio, (i.e., —S—CH₃).

“Alkoxy” refers to an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy.

“Alkylamino” refers to an alkyl group as defined above with the indicated number of carbon atoms attached through an amino bridge. An example of an alkylamino is methylamino, (i.e., —NH—CH₃).

“Dialkylamino” refers to two alkyl group as defined above with the indicated number of carbon atoms attached through an amino bridge. An example of an alkylamino is dimethylamino, (i.e., −N(CH₃)₂.

The terms “halogen” and “halo” refer to fluorine, chlorine, bromine, and iodine.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing a oxygen atom with a sulfur atom or replacing an amino group with a hydroxyl group. The derivative may be a prodrug. Derivatives may be prepared by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“=O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRₐR_{b}, —NR^{a}C(═O)R_{b}, —NRₐC(═O)NRₐNR_{b}, —NRₐC(═O)OR_{b}, —NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ and —S(═O)₂ORₐ. Rₐ and R_{b} in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

The term “optionally substituted,” as used herein, means that substitution is optional and therefore it is possible for the designated atom to be unsubstituted.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Typical prodrugs are pharmaceutically acceptable esters. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In certain embodiment the salts are conventional nontoxic pharmaceutically acceptable salts including the quaternary ammonium salts of the parent compound formed, and non-toxic inorganic or organic acids. Contemplated salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, maleic, hydroxymaleic, phenylacetic, glutamic, benzoic, salicylic, sulfanilic, 2-acetoxybenzoic, toluenesulfonic, methanesulfonic, ethane disulfonic, oxalic, isethionic, and the like.

“Subject” refers any animal, preferably a human patient, livestock, or domestic pet.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g. patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

### COMPOUNDS

In certain embodiments, the disclosure relates to a compound or pharmaceutical composition comprising a compound of formula I,

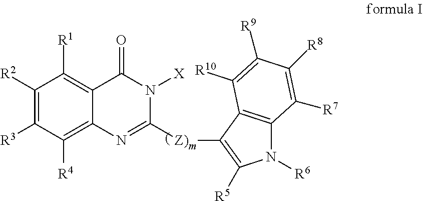

or pharmaceutically acceptable salt or prodrug thereof wherein,
X is —(Q)ₙ—Y;
n is 1, 2, 3, 4 or 5;
m is 1, 2, 3, 4 or 5;
Q is at each occurrence, individually and independently, O, S, NH, C═O, CH₂, or CH═CH;
Y is dialkylamine, carbocyclyl, aryl, or heterocyclyl optionally substituted with one or more, the same or different, R¹¹,
Z is at each occurrence, individually and independently, O, S, NH, C═O, CH₂, or CH═CH;
R¹, R², R³, R⁴, R⁵, R⁶, R⁷, R⁸, R⁹ and R¹⁰ are each individually and independently hydrogen, alkyl, halogen, nitro, cyano, hydroxy, amino, mercapto, formyl, carboxy, carbamoyl, alkoxy, alkylthio, alkylamino, (alkyl)₂amino, carbocyclyl, aryl, or heterocyclyl, wherein R¹, R², R³, R⁴, R⁵, R⁶, R⁷, R⁸, R⁹ and R¹⁰ are optionally substituted with one or more, the same or different, R¹¹;
R¹¹ is alkyl, halogen, nitro, cyano, hydroxy, amino, mercapto, formyl, carboxy, carbamoyl, alkoxy, alkylthio, alkylamino, (alkyl)zamino, carbocyclyl, aryl, or heterocyclyl, wherein R¹¹ is optionally substituted with one or more, the same or different, R¹²; and
R¹² is halogen, nitro, cyano, hydroxy, trifluoromethoxy, trifluoromethyl, amino, formyl, carboxy, carbamoyl, mercapto, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methoxycarbonyl, ethoxycarbonyl, carbocyclyl, aryl, or heterocyclyl.

In certain embodiments, R¹, R³, R⁴, R⁵, R⁶, R⁷, R⁸, R⁹ and R¹⁰ are each individually and independently hydrogen, alkyl, halogen, nitro, cyano, hydroxy, amino, mercapto, formyl, carboxy, carbamoyl, alkoxy, alkylthio, alkylamino, (alkyl)zamino, carbocyclyl, aryl, or heterocyclyl, wherein R¹, R³, R⁴, R⁵, R⁶, R⁷, R⁸, R⁹ and R¹⁰ are optionally substituted with one or more, the same or different, R¹¹; and R² is halogen.

In certain embodiments, R² is halogen.

In certain embodiments, R² is hydrogen.

In certain embodiments, R² is halogen or hydrogen.

Examples compounds include 2-((2-(1H-indol-3-yl)ethyl)thio)-3-(3-(dimethylamino)propyl)-6-fluoroquinazolin-4(3H)-one and 2-((2-(1H-indol-3-yl)ethyl)thio)-6-chloro-3-(3-(dimethylamino)propyl)quinazolin-4(3H)-one, salts, prodrugs, and derivatives thereof.

In certain embodiments, the disclosure relates to a compound or pharmaceutical composition comprising a compound of formula II or formula IIA,

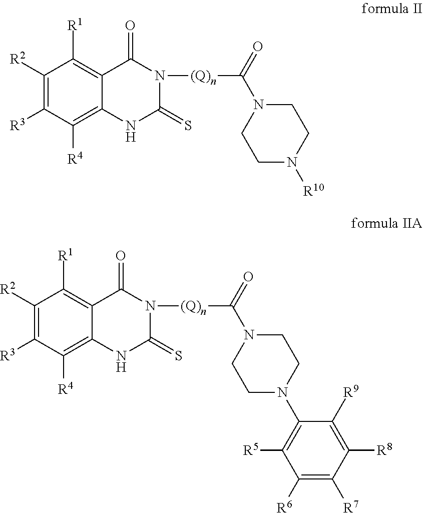

or pharmaceutically acceptable salt or prodrug thereof wherein,
n is 1, 2, 3, 4 or 5;
Q is at each occurrence, individually and independently, O, S, NH, C═O, CH₂, or CH═CH;
R¹, R², R³, R⁴, R⁵, R⁶, R⁷, R⁸, and R⁹ are each individually and independently hydrogen, alkyl, halogen, nitro, cyano, hydroxy, amino, mercapto, formyl, carboxy, carbamoyl, alkoxy, alkylthio, alkylamino, (alkyl)zamino, carbocyclyl, aryl, or heterocyclyl, wherein R¹, R², R³, R⁴, R⁵, R⁶, R⁷, R⁸, and R⁹ are optionally substituted with one or more, the same or different, R¹⁰;
R¹⁰ is alkyl, halogen, nitro, cyano, hydroxy, amino, mercapto, formyl, carboxy, carbamoyl, alkoxy, alkylthio, alkylamino, (alkyl)zamino, carbocyclyl, aryl, or heterocyclyl, wherein R¹⁰ is optionally substituted with one or more, the same or different, R¹¹; and
R¹¹ is halogen, nitro, cyano, hydroxy, trifluoromethoxy, trifluoromethyl, amino, formyl, carboxy, carbamoyl, mercapto, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methoxycarbonyl, ethoxycarbonyl, carbocyclyl, aryl, or heterocyclyl.

In certain embodiments, R² is halogen.

In certain embodiments, R⁵ is hydroxy optionally substituted with one or more R¹⁰.

An example compound is 6-fluoro-3-(4-(4-(2-hydroxyphenyl)piperazin-1-yl)-4-oxobutyl)-2-thioxo-2,3-dihydroquinazolin-4(1H)-one, salts, prodrugs, and derivatives thereof.

In certain embodiments, the disclosure relates to a compound or pharmaceutical composition comprising a compound of formula III,

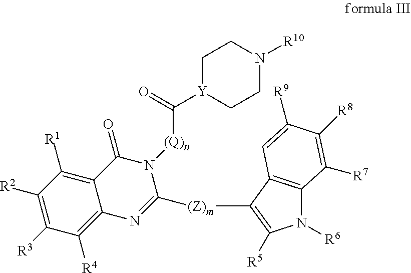

or pharmaceutically acceptable salt or prodrug thereof wherein,
X is —(Q)ₙ—Y;
n is 1, 2, 3, 4 or 5;
m is 1, 2, 3, 4 or 5;
Q is at each occurrence, individually and independently, O, S, NH, C═O, CH₂, or CH═CH;
Y is dialkylamine, carbocyclyl, aryl, or heterocyclyl optionally substituted with one or more, the same or different, R¹¹;
Z is at each occurrence, individually and independently, O, S, NH, C═O, CH₂, or CH═CH;
R¹, R², R³, R⁴, R⁵, R⁶, R⁷, R⁸, R⁹ and R¹⁰ are each individually and independently hydrogen, alkyl, halogen, nitro, cyano, hydroxy, amino, mercapto, formyl, carboxy, carbamoyl, alkoxy, alkylthio, alkylamino, (alkyl)₂amino, carbocyclyl, aryl, or heterocyclyl, wherein R¹, R², R³, R⁴, R⁵, R⁶ , R⁷, R⁸, R⁹ and R¹⁰ are optionally substituted with one or more, the same or different, R¹¹;
R¹¹ is alkyl, halogen, nitro, cyano, hydroxy, amino, mercapto, formyl, carboxy, carbamoyl, alkoxy, alkylthio, alkylamino, (alkyl)zamino, carbocyclyl, aryl, or heterocyclyl, wherein R¹¹ is optionally substituted with one or more, the same or different, R¹²; and
R¹² is halogen, nitro, cyano, hydroxy, trifluoromethoxy, trifluoromethyl, amino, formyl, carboxy, carbamoyl, mercapto, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methoxycarbonyl, ethoxycarbonyl, carbocyclyl, aryl, or heterocyclyl.

In certain embodiments, R² is halogen.

In certain embodiments, R² is hydrogen.

In certain embodiments, R² is halogen or hydrogen.

### Formulations

Pharmaceutical compositions disclosed herein comprise a compound disclosed herein and a pharmaceutically acceptable excipient. The compound may be in the form of a pharmaceutically acceptable salt, as generally described below. Some preferred, but non-limiting examples of suitable pharmaceutically acceptable inorganic acids are hydrochloric acid, hydrobromic acid, sulfuric acid, nitric acid, acetic acid and citric acid, as well as other pharmaceutically acceptable acids known per se (for which reference is made to the references referred to below).

When the compounds of the disclosure contain an acidic group as well as a basic group, the compounds of the disclosure may also form internal salts, and such compounds are within the scope of the disclosure. When a compound contains a hydrogen-donating heteroatom (e.g. NH), salts are contemplated to covers isomers formed by transfer of said hydrogen atom to a basic group or atom within the molecule.

Pharmaceutically acceptable salts of the compounds include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

The compounds described herein may be administered in the form of prodrugs. A prodrug can include a covalently bonded carrier, which releases the active parent drug when administered to a mammalian subject. Prodrugs can be prepared by modifying functional groups present in the compounds in such a way that the modifications are cleaved, either in routine manipulation or in vivo, to the parent compounds. Prodrugs include, for example, compounds wherein a hydroxyl group is bonded to any group that, when administered to a mammalian subject, cleaves to form a free hydroxyl group. Examples of prodrugs include, but are not limited to, acetate, formate and benzoate derivatives of alcohol functional groups in the compounds. Methods of structuring a compound as prodrugs can be found in the book of Testa and Mayer, Hydrolysis in Drug and Prodrug Metabolism, Wiley (2006). Typical prodrugs form the active metabolite by transformation of the prodrug by hydrolytic enzymes, the hydrolysis of amide, lactams, peptides, carboxylic acid esters, epoxides or the cleavage of esters of inorganic acids.

Pharmaceutical compositions for use in the present disclosure typically comprise an effective amount of a compound and a suitable pharmaceutical acceptable carrier. The preparations may be prepared in a manner known per se, which usually involves mixing the at least one compound according to the disclosure with the one or more pharmaceutically acceptable carriers, and, if desired, in combination with other pharmaceutical active compounds, when necessary under aseptic conditions. Reference is again made to U.S. Pat. Nos. 6,372,778; 6,369,086; 6,369,087; 6,372,733 and the further references mentioned above, as well as to the standard handbooks, such as the latest edition of Remington's Pharmaceutical Sciences.

Generally, for pharmaceutical use, the compounds may be formulated as a pharmaceutical preparation comprising at least one compound and at least one pharmaceutically acceptable carrier, diluent or excipient and/or adjuvant, and optionally one or more further pharmaceutically active compounds.

The pharmaceutical preparations of the disclosure are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of the at least one compound of the disclosure, e.g. about 10, 25, 50, 100, 200, 300 or 400 mg per unit dosage.

The compounds can be administered by a variety of routes including the oral, ocular, rectal, transdermal, subcutaneous, intravenous, intramuscular, or intranasal routes, depending mainly on the specific preparation used. The compound will generally be administered in an “effective amount”, by which is meant any amount of a compound that, upon suitable administration, is sufficient to achieve the desired therapeutic or prophylactic effect in the subject to which it is administered. Usually, depending on the condition to be prevented or treated and the route of administration, such an effective amount will usually be between 0.01 to 1000 mg per kilogram body weight of the patient per day, more often between 0.1 and 500 mg, such as between 1 and 250 mg, for example about 5, 10, 20, 50, 100, 150, 200 or 250 mg, per kilogram body weight of the patient per day, which may be administered as a single daily dose, divided over one or more daily doses. The amount(s) to be administered, the route of administration and the further treatment regimen may be determined by the treating clinician, depending on factors such as the age, gender and general condition of the patient and the nature and severity of the disease/symptoms to be treated. Reference is again made to U.S. Pat. Nos. 6,372,778; 6,369,086; 6,369,087; and 6,372,733 and the further references mentioned above, as well as to the standard handbooks, such as the latest edition of Remington's Pharmaceutical Sciences.

Depending upon the manner of introduction, the compounds described herein may be formulated in a variety of ways. Formulations containing one or more Nox inhibitors can be prepared in various pharmaceutical forms, such as granules, tablets, capsules, suppositories, powders, controlled release formulations, suspensions, emulsions, creams, gels, ointments, salves, lotions, or aerosols and the like. Preferably, these formulations are employed in solid dosage forms suitable for simple, and preferably oral, administration of precise dosages. Solid dosage forms for oral administration include, but are not limited to, tablets, soft or hard gelatin or non-gelatin capsules, and caplets. However, liquid dosage forms, such as solutions, syrups, suspension, shakes, etc. can also be utilized. In another embodiment, the formulation is administered topically. Suitable topical formulations include, but are not limited to, lotions, ointments, creams, and gels. In a preferred embodiment, the topical formulation is a gel. In another embodiment, the formulation is administered intranasally.

Formulations containing one or more of the compounds described herein may be prepared using a pharmaceutically acceptable carrier composed of materials that are considered safe and effective and may be administered to an individual without causing undesirable biological side effects or unwanted interactions. The carrier is all components present in the pharmaceutical formulation other than the active ingredient or ingredients. As generally used herein “carrier”includes, but is not limited to, diluents, binders, lubricants, disintegrators, fillers, pH modifying agents, preservatives, antioxidants, solubility enhancers, and coating compositions.

Carrier also includes all components of the coating composition which may include plasticizers, pigments, colorants, stabilizing agents, and glidants. Delayed release, extended release, and/or pulsatile release dosage formulations may be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants.

Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropyl ethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, or cross linked polymers.

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine, and lauryl sulfobetaine.

If desired, the tablets, beads, granules, or particles may also contain minor amount of nontoxic auxiliary substances such as wetting or emulsifying agents, dyes, pH buffering agents, or preservatives.

The concentration of the Nox inhibitor(s) to carrier and/or other substances may vary from about 0.5 to about 100 wt % (weight percent). For oral use, the pharmaceutical formulation will generally contain from about 5 to about 100% by weight of the active material. For other uses, the pharmaceutical formulation will generally have from about 0.5 to about 50 wt. % of the active material.

The compositions described herein can be formulation for modified or controlled release. Examples of controlled release dosage forms include extended release dosage forms, delayed release dosage forms, pulsatile release dosage forms, and combinations thereof.

The extended release formulations are generally prepared as diffusion or osmotic systems, for example, as described in “Remington—The science and practice of pharmacy” (20th ed., Lippincott Williams & Wilkins, Baltimore, MD, 2000). A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such hydroxypropyl-cellulose, hydroxypropyl ethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets andcapsules containing tablets, beads, or granules An immediate release portion can be added to the extended release system by means of either applying an immediate release layer on top of the extended release core using a coating or compression process or in a multiple unit system such as a capsule containing extended and immediate release beads. Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient.

The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is typical in a tablet formulation to prevent the tablet and punches from sticking in the die. The lubricant is chosen from such slippery solids as talc, magnesium and calcium stearate, stearic acid and hydrogenated vegetable oils.

The Nox inhibitors described herein can be administered adjunctively with other active compounds. These compounds include but are not limited to analgesics, anti-inflammatory drugs, antipyretics, antidepressants, antiepileptics, antihistamines, antimigraine drugs, antimuscarinics, anxiolytics, sedatives, hypnotics, antipsychotics, bronchodilators, anti-asthma drugs, cardiovascular drugs, corticosteroids, dopaminergics, electrolytes, gastro-intestinal drugs, muscle relaxants, nutritional agents, vitamins, parasympathomimetics, stimulants, anorectics and anti-narcoleptics. “Adjunctive administration”, as used herein, means the Nox inhibitors can be administered in the same dosage form or in separate dosage forms with one or more other active agents.

Specific examples of compounds that can be adjunctively administered with the Nox inhibitors include, but are not limited to, aceclofenac, acetaminophen, atomoxetine, almotriptan, alprazolam, amantadine, amcinonide, aminocyclopropane, amitriptyline, amlodipine, amphetamine, aripiprazole, aspirin, atomoxetine, azasetron, azatadine, beclomethasone, benactyzine, benoxaprofen, bermoprofen, betamethasone, bicifadine, bromocriptine, budesonide, buprenorphine, bupropion, buspirone, butorphanol, butriptyline, caffeine, carbamazepine, carbidopa, carisoprodol, celecoxib, chlordiazepoxide, chlorpromazine, choline salicylate, citalopram, clomipramine, clonazepam, clonidine, clonitazene, clorazepate, clotiazepam, cloxazolam, clozapine, codeine, corticosterone, cortisone, cyclobenzaprine, cyproheptadine, demexiptiline, desipramine, desomorphine, dexamethasone, dexanabinol, dextroamphetamine sulfate, dextromoramide, dextropropoxyphene, dezocine, diazepam, dibenzepin, diclofenac sodium, dihydromorphinone, divalproex, dizatriptan, dolasetron, donepezil, dothiepin, doxepin, duloxetine, ergotamine, escitalopram, estazolam, ethosuximide, etodolac, femoxetine, fenamates, fenoprofen, fentanyl, fludiazepam, fluoxetine, fluphenazine, flurazepam, flurbiprofen, flutazolam, fluvoxamine, frovatriptan, gabapentin, galantine, gepirone, gingko balboa, granisetron, haloperidol, huperzine A, hydrocodone, hydrocortisone, hydromorphone, hydroxyzine, ibuprofen, imipramine, indiplon, indomethacin, indoprofen, ipsapirone, ketanserin, ketorolac, lesopitron, levodopa, lipase, lofepramine, lorazepam, loxapine, maprotiline, mazindol, mefenamic acid, melatonin, melitracen, memantine, meperidine, meprobamate, mesalamine, metapramine, metaxalone, methadone, methadone, methamphetamine, methocarbamol, methyldopa, methylphenidate, methyl salicylate, methysergide, metoclopramide, mianserin, mifepristone, milnacipran, minaprine, mirtazapine, moclobemide, modafinil (an anti-narcoleptic), molindone, morphine, morphine hydrochloride, nabumetone, nadolol, naproxen, naratriptan, nefazodone, neurontin, nomifensine, nortriptyline, olanzapine, olsalazine, ondansetron, oxaflozane, oxaprozin, oxitriptan, oxycodone, pancrelipase, paroxetine, etazocine, pepsin, phenacetin, phenytoin, phosphatidylserine, prednisolone, prednisone, propranolol, propoxyphene, nortriptyline, reserpine, risperidone, ritanserin, dizatriptan, rofecoxib, rotigotine, salsalate, sertraline, sildenafil, sulfasalazine, sulfidic, thiazides, tramadol, trazodone, triazole, imipramine, tropisetron, valproic acid, venlafaxine, vitamin E, zimeldine, ziprasidone, zolmitriptan, zolpidem, zopiclone and isomers, salts, and combinations thereof.

### EXPERIMENTAL

### Synthesis of comnaunds

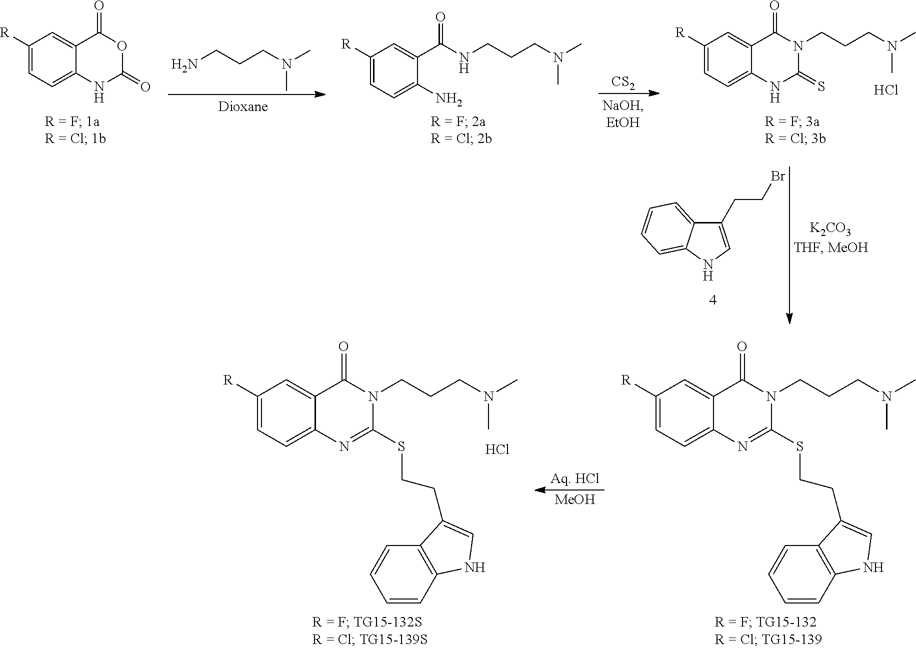

### 2-((2-(1H-indol-3-yl)ethyl)thio)-3-(3-(dimethylamino)propyl)-6-fluoroquinazolin-4(3H)-one, hydrochloride (TG15-132S)

¹H NMR (400 MHZ, DMSO-d6): δ 10.95 (s, 1H), 10.11 (s, 1H), 7.80-7.64 (m, 4H), 7.36 (d, J=8.0 Hz, 1H), 7.28 (d, J=2.2 Hz, 1H), 7.13-7.00 (m, 2H), 4.13 (t, J=7.0 Hz, 2H), 3.58 (t, J=7.5 Hz, 2H), 3.20-3.09 (m, 4H), 2.72 (s, 6H), 2.14-2.04 (m, 2H); 19F NMR (125 MHz) δ-114.9 (m); LCMS (ESI): LCMS (ESI): >95% purity; m/z, 425 [(M-HCI)+H]+; HPLC purity: 96.4%.

### 2-((2-(1H-indol-3-yl)ethyl)thio)-6-chloro-3-(3-(dimethylamino)propyl)quinazolin-4(3H)-one. Hydrochloride (TG15-139S)

¹H NMR (400 MHZ, DMSO-d6): δ 10.95 (s, 1H), 10.24 (s, 1H), 8.03 (d, J=2.5 Hz, 1H), 7.86 (dd, J=8.7, 2.5 Hz, 1H), 7.73-7.58 (m, 2H), 7.36 (d, J=7.9 Hz, 1H), 7.28 (d, J=2.0 Hz, 1H), 7.13-6.98 (m, 2H), 4.12 (t, J=6.9 Hz, 2H), 3.67-3.48 (m, 2H), 3.25-3.06 (m, 4H), 2.71 (s, 6H), 2.20-1.99 (m, 2H); LCMS (ESI): LCMS (ESI): >95% purity; m/z, 441 [(M-HCI)+H]+; HPLC purity: 95.7%.

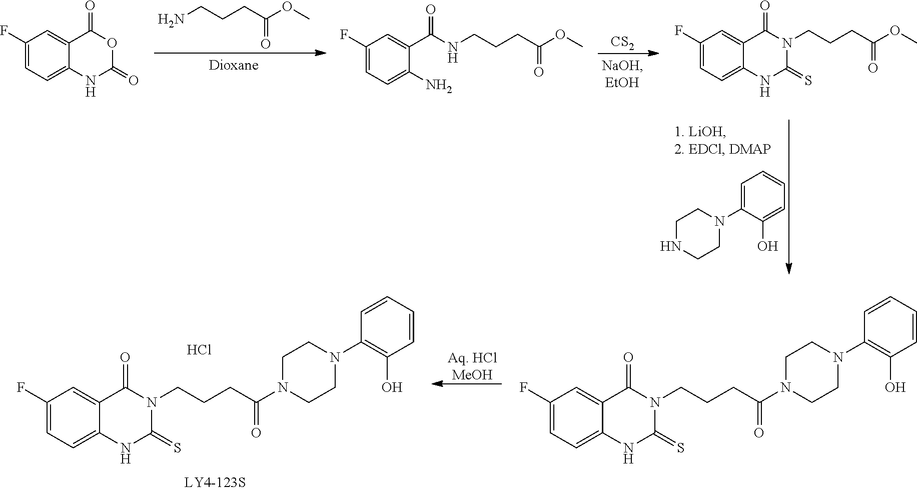

### Drug Metabolism and Pharmacokinetics (DMPK)

Several physicochemical properties were calculated for the compounds shown in table 1. below.

TABLE 1

| | | | PTSA | | | Water | MLM | In vivo |
|---|---|---|---|---|---|---|---|---|
| Compd. | M.W. | cLogP | (Å) | pKa | HBD | Solubility | T_{1/2} | B/P Ratio |
| TG15-132S | 424 | 4.6 | 47.9 | 9.2 | 2 | 1.2 mM | 24 min | >10 |
| TG15-139S | 440 | 5.1 | 47.9 | 9.2 | 2 | 2.35 mM | 75 min | IP |
| LY4-1238 | 442 | 2.25 | 76.1 | 9.6 | 2 | IP | IP | IP |

The data indicated that these compounds may have CNS permeability and activity based on their molecular weight and total polar surface area (<80 \acute{Å}). The aqueous solubility, liver microsomal stability, and in vivo pharmacokinetics were determined. TG15-132S and TG15-139S have excellent water solubility, which enable us to design formulations for oral and other methods of delivery. These derivatives have requisite stability in mouse liver microsomes. Furthermore, replacement of fluorine with chlorine has dramatically improved the water solubility and liver microsomal stability.

### In Vivo Pharmacokinetics for Compound TG15-132S in Sprague Dawley (SD) Rats.

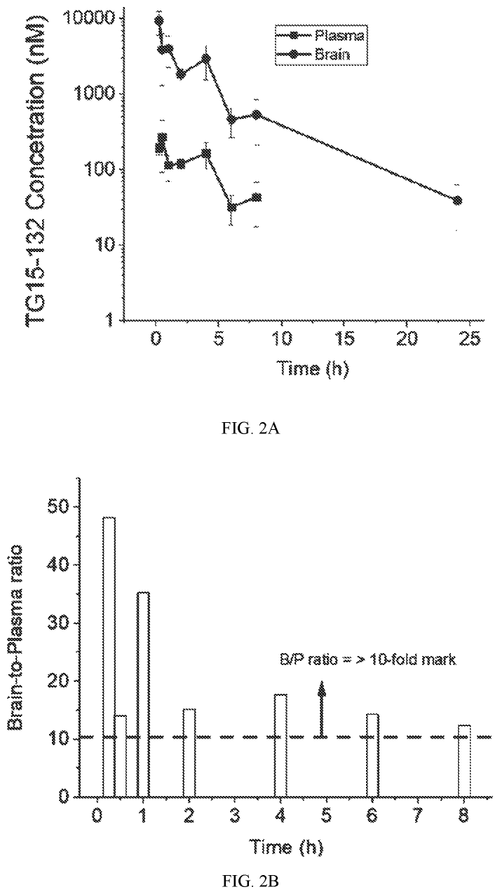

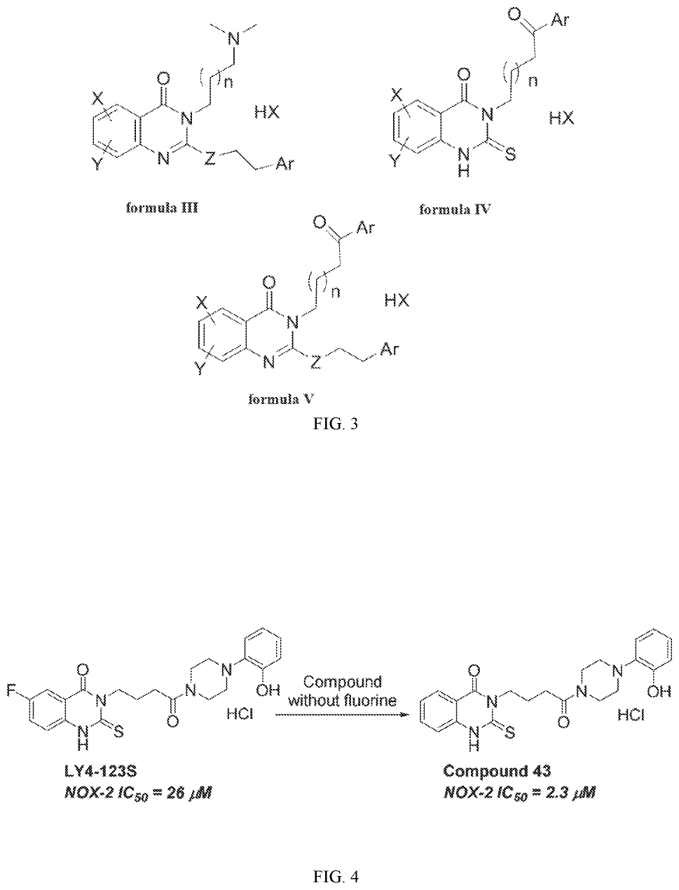

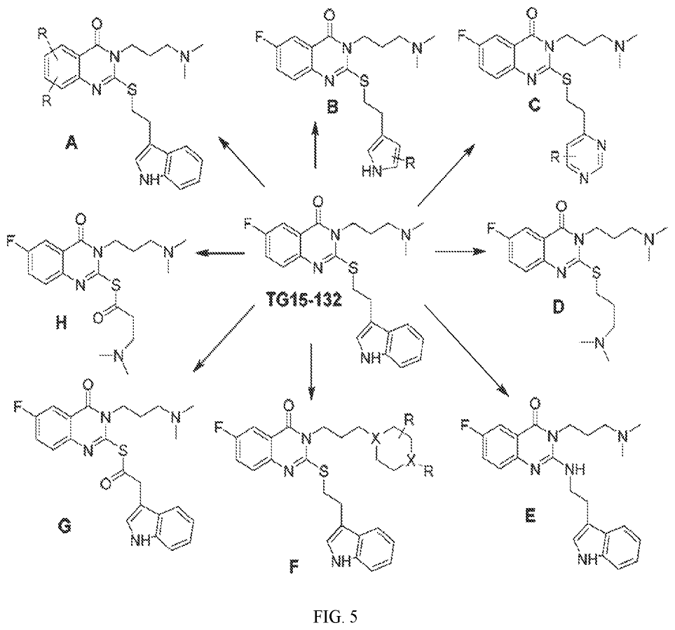

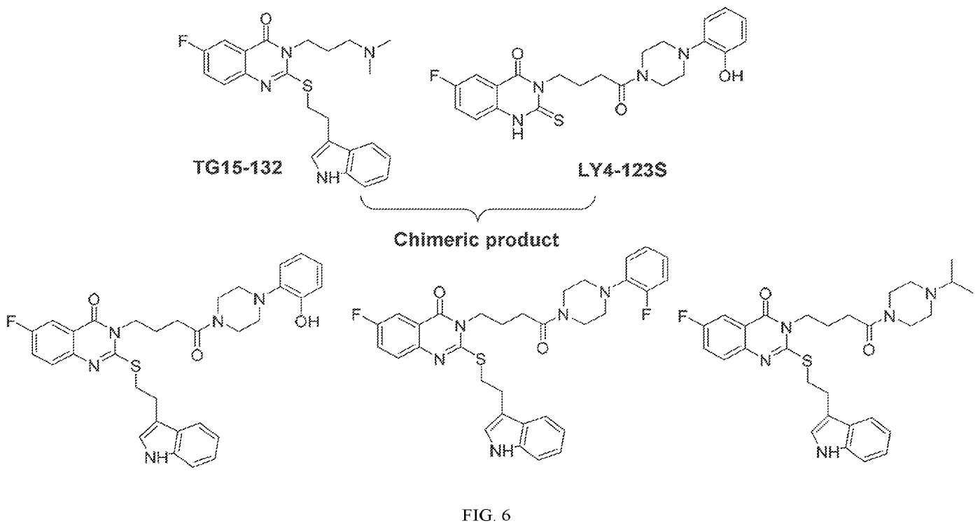

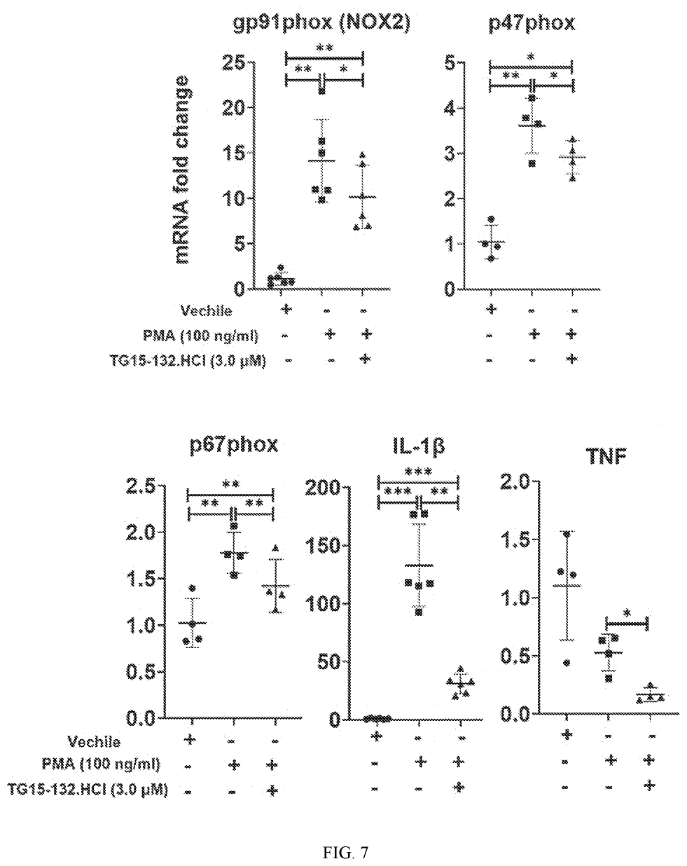

As shown in FIG. 2, after single dosing of 20 mg/kg by intraperitoneal injection, the plasma and brain half-life>5 h. AUC_{inf} of 10180 (hr*ng/ml) and clearance rate of 32.75 (mL/min/kg). Interestingly the brain-to-plasma ratio of TG15-139S is>23-fold (>10-fold up to 8 h).

TABLE 2
Additional Examples

| | | | NOX-2 | NOX-1 | NOX-4 | |
|---|---|---|---|---|---|---|
| | | MW/ | IC₅₀ | IC₅₀ | IC₅₀ | CC₅₀ |
| | Structure | FW | (μM) | (μM) | (μM) | (μM) |
| TG15- 131- HCl | 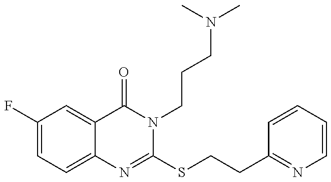 [SMILES: CN(C)CCCn1c(SCCc2ccccn2)nc2ccc(F)cc2c1=O] | 422 | >50 | | | ND |
| | 3-(3-(dimethylamino)propyl)-6- | | | | | |
| | fluoro-2-((2-(pyridin-2- | | | | | |
| | yl)ethyl)thio)quinazolin-4(3H)- | | | | | |
| | one | | | | | |
| TG15- 132- HCl | 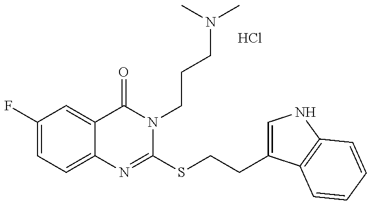 [SMILES: CN(C)CCCn1c(SCCc2c[nH]c3ccccc23)nc2ccc(F)cc2c1=O ; Cl] | 460 | 3.3 | | 26 | >20 |
| | 2-((2-(1H-indol-3-yl)ethyl)thio)- | | | | | |
| | 3-(3-(dimethylamino)propyl)-6- | | | | | |
| | fluoroquinazolin-4(3H)-one | | | | | |
| TG15- 124- HCl | 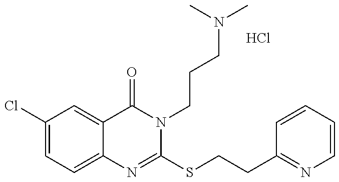 [SMILES: CN(C)CCCn1c(SCCc2ccccn2)nc2ccc(Cl)cc2c1=O ; Cl] | 438 | >100 | | | >10 |
| | 6-chloro-3-(3- | | | | | |
| | (dimethylamino)propyl)-2-((2- | | | | | |
| | (pyridin-2- | | | | | |
| | yl)ethyl)thio)quinazolin-4(3H)- | | | | | |
| | one | | | | | |
| TG15- 139- HCl | 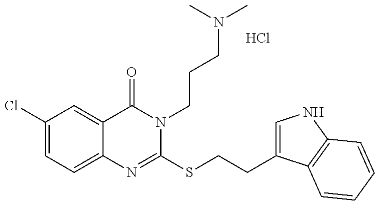 [SMILES: CN(C)CCCn1c(SCCc2c[nH]c3ccccc23)nc2ccc(Cl)cc2c1=O ; Cl] | 476 | 1.65 | | 32 | >10 |
| | 2-((2-(1H-indol-3-yl)ethyl)thio)- | | | | | |
| | 6-chloro-3-(3- | | | | | |
| | (dimethylamino)propyl)quinazolin- | | | | | |
| | 4(3H)-one | | | | | |
| LY5- 10 | 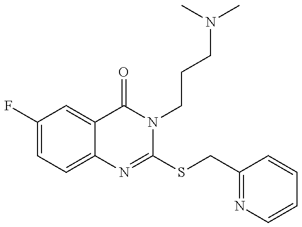 [SMILES: CN(C)CCCn1c(SCc2ccccn2)nc2ccc(F)cc2c1=O] | 372 | >100 | | | |
| | 3-(3-(dimethylamino)propyl)-6- | | | | | |
| | fluoro-2-((pyridin-2- | | | | | |
| | ylmethyl)thio)quinazolin-4(3H)- | | | | | |
| | one | | | | | |
| LY5- 11 | 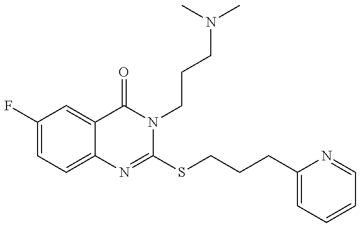 [SMILES: CN(C)CCCn1c(SCCCc2ccccn2)nc2ccc(F)cc2c1=O] | 400 | >100 | | | |
| | 3-(3-(dimethylamino)propyl)-6- | | | | | |
| | fluoro-2-((3-(pyridin-2- | | | | | |
| | yl)propyl)thio)quinazolin-4(3H)- | | | | | |
| | one | | | | | |
| LY4- 123S (TG17-29) | 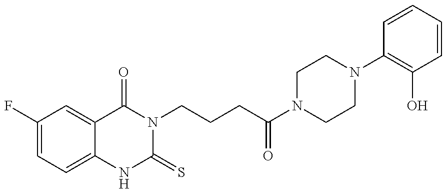 [SMILES: O=C(CCCn1c(=S)[nH]c2ccc(F)cc2c1=O)N1CCN(c2ccccc2O)CC1] | 442 | 3.3 | | 8.3 | |
| | 6-fluoro-3-(4-(4-(2- | | | | | |
| | hydroxyphenyl)piperazin-1-yl)- | | | | | |
| | 4-oxobutyl)-2-thioxo-2,3- | | | | | |
| | dihydroquinazolin-4(1H)-one | | | | | |
| Compd. 43 | 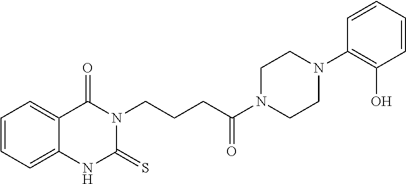 [SMILES: O=C(CCCn1c(=S)[nH]c2ccccc2c1=O)N1CCN(c2ccccc2O)CC1] | 424 | 2.3 | | >50 | >20 |
| | 3-(4-(4-(2- | | | | | |
| | hydroxyphenyl)piperazin-1-yl)- | | | | | |
| | 4-oxobutyl)-2-thioxo-2,3- | | | | | |
| | dihydroquinazolin-4(1H)-one | | | | | |
| TG15- 288 | 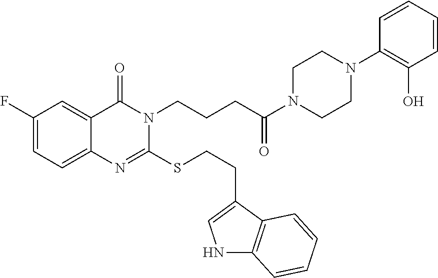 [SMILES: O=C(CCCn1c(SCCc2c[nH]c3ccccc23)nc2ccc(F)cc2c1=O)N1CCN(c2ccccc2O)CC1] | 585 | IP | | | |
| | 2-((2-(1H-indol-3-yl)ethyl)thio)- | | | | | |
| | 6-fluoro-3-(4-(4-(2- | | | | | |
| | hydroxyphenyl)piperazin-1-yl)- | | | | | |
| | 4-oxobutyl)quinazolin-4(3H)- | | | | | |
| | one | | | | | |
| TG15- 289 | 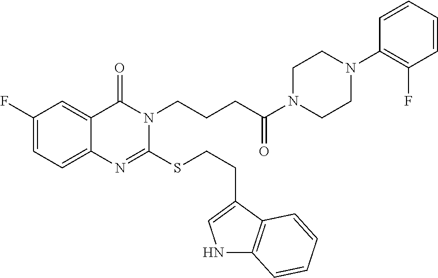 [SMILES: O=C(CCCn1c(SCCc2c[nH]c3ccccc23)nc2ccc(F)cc2c1=O)N1CCN(c2ccccc2F)CC1] | 587 | IP | | | |
| | 2-((2-(1H-indol-3-yl)ethyl)thio)- | | | | | |
| | 6-fluoro-3-(4-(4-(2- | | | | | |
| | fluorophenyl)piperazin-1-yl)-4- | | | | | |
| | oxobutyl)quinazolin-4(3H)-one | | | | | |
| TG15- 293 | 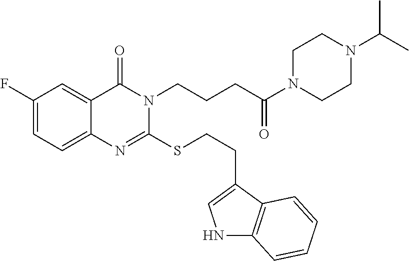 [SMILES: CC(C)N1CCN(C(=O)CCCn2c(SCCc3c[nH]c4ccccc34)nc3ccc(F)cc3c2=O)CC1] | 535 | IP | | | |
| TG17- 55-Salt | 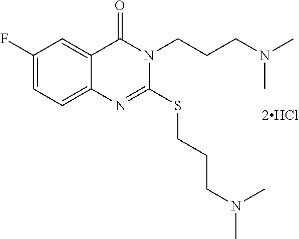 [SMILES: CN(C)CCCSc1nc2ccc(F)cc2c(=O)n1CCCN(C)C] | 438 | IP | | | |
| | 2-((2-(1H-indol-3-yl)ethyl)thio)- | | | | | |
| | 6-fluoro-3-(4-(4- | | | | | |
| | isopropylpiperazin-1-yl)-4- | | | | | |
| | oxobutyl)quinazolin-4(3H)-one | | | | | |
| TG17- 56 | 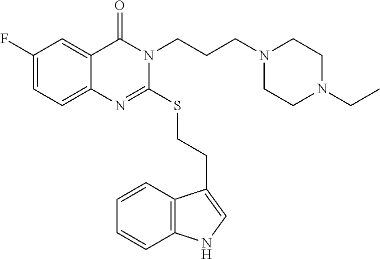 [SMILES: CCN1CCN(CCCn2c(SCCc3c[nH]c4ccccc34)nc3ccc(F)cc3c2=O)CC1] | 493 | IP | | | |
| | 2-((2-(1H-indol-3-yl)ethyl)thio)- | | | | | |
| | 3-(3-(4-ethylpiperazin-1- | | | | | |
| | yl)propyl)-6-fluoroquinazolin- | | | | | |
| | 4(3H)-one | | | | | |
| TG17- 57 | 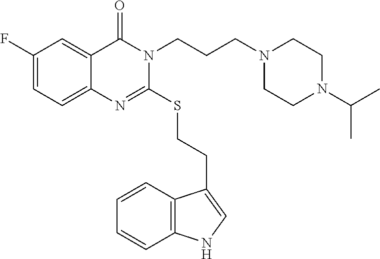 [SMILES: CC(C)N1CCN(CCCn2c(SCCc3c[nH]c4ccccc34)nc3ccc(F)cc3c2=O)CC1] | 507 | 2.4 | | | |
| | 2-((2-(1H-indol-3-yl)ethyl)thio)- | | | | | |
| | 6-fluoro-3-(3-(4- | | | | | |
| | isopropylpiperazin-1- | | | | | |
| | yl)propyl)quinazolin-4(3H)-one | | | | | |

What is claimed is:

1. A compound of formula I, 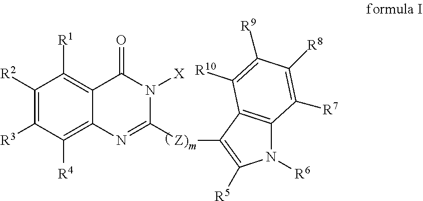
or pharmaceutically acceptable salt or prodrug thereof wherein,
X is —(Q)ₙ—Y;
n is 1, 2, 3, 4 or 5;
m is 1, 2, 3, 4 or 5;
Q is at each occurrence, individually and independently, O, S, NH, C═O, CH₂, or CH═CH;
Y is dialkylamine, carbocyclyl, aryl, or heterocyclyl optionally substituted with one or more, the same or different, R¹¹;
Z is at each occurrence, individually and independently, O, S, NH, C═O, or CH₂;
R¹, R³, R⁴, R⁵, R⁶, R⁷, R⁸, R⁹ and R¹⁰ are each individually and independently hydrogen, alkyl, halogen, nitro, cyano, hydroxy, amino, mercapto, formyl, carboxy, carbamoyl, alkoxy, alkylthio, alkylamino, (alkyl)zamino, carbocyclyl, aryl, or heterocyclyl, wherein R¹, R³, R⁴, R⁵, R⁶, R⁷, R⁸, R⁹ and R¹⁰ are optionally substituted with one or more, the same or different, R¹¹;
R² is halogen;
R¹¹ is alkyl, halogen, nitro, cyano, hydroxy, amino, mercapto, formyl, carboxy, carbamoyl, alkoxy, alkylthio, alkylamino, (alkyl)zamino, carbocyclyl, aryl, or heterocyclyl, wherein R¹¹ is optionally substituted with one or more, the same or different, R¹²; and
R¹² is halogen, nitro, cyano, hydroxy, trifluoromethoxy, trifluoromethyl, amino, formyl, carboxy, carbamoyl, mercapto, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methoxycarbonyl, ethoxycarbonyl, carbocyclyl, aryl, or heterocyclyl.

2. The compound of claim 1 which is 2-((2-(1H-indol-3-yl)ethyl)thio)-3-(3-(dimethylamino)propyl)-6-fluoroquinazolin-4(3H)-one or salt thereof.

3. The compound of claim 1 which is 2-((2-(1H-indol-3-yl)ethyl)thio)-6-chloro-3-(3-(dimethylamino)propyl)quinazolin-4(3H)-one or salt thereof.

4. A pharmaceutical composition comprising a compound of claim 1 and a pharmaceutically acceptable excipient or carrier.

5. The pharmaceutical composition of claim 4 in the form of a pill, capsule, tablet, gel, granule, or aqueous phosphate buffer solution.

6. A method of treating a Nox related disease comprising administering a composition of claim 1 to a subject in need thereof.

7. The method of claim 6, wherein the Nox related disease is selected from traumatic brain injury and epilepsy.

8. (canceled)

9. A method of treating a central nervous system disorder comprising administering a composition comprising a compound of claim 1 to a subject in need thereof.

10. A method of preventing tissue injury comprising administering a compound of claim 1 to subject before, during or after and organ transplant.

11. An aqueous biological storage medium comprising a compound of claim 1 and further comprising an amino acid, a saccharide, a vitamin, or adenosine triphosphate.
